(19) United States
(12) Patent Application Publication
(10) Pub. No.: US 2008/0306563 A1
(43) Pub. Date: Dec. 11, 2008

(54) System and method for cardiovascular treatment or training

(76) Inventors: Jose Roberto Kullok, Jerusalem (IL); Saul Kullok, Jerusalem (IL)

Correspondence Address: THE NATH LAW GROUP, 112 South West Street, Alexandria, VA, 22314, US

(21) Appl. No.: 11/806,987

(22) Filed: Jun. 5, 2007

(51) Int. Cl. A61N 1/36

(52) U.S. Cl. 607/17; 607/23

(57) ABSTRACT

The invention provides a system and method for cardiovascular treatment or training of an individual. One or more venous return devices are used to alter the venous return in an individual, and one or more heart rate devices are used to alter the individual's heart rate. A processor activates the venous return devices and the one or more heart rate devices at one or more predetermined levels for one or more predetermined time durations

### FIELD OF THE INVENTION

This invention relates to medical devices, and more specifically, to such devices for cardiovascular treatment and training.

### BACKGROUND OF THE INVENTION

Human hypertension affects one billion people worldwide, and is implicated in 7.1 million deaths each year, resulting from ischemic heart disease and stroke alone. From family and epidemiological studies, it appears that hypertension arises from a complex interplay between genetic, environmental and lifestyle factors. Approximately 95% of the cases consist in primary or essential hypertension for which no full understanding exists. The other 5% of the cases is a secondary hypertension, where the cause is recognized to be due to diseases generally arising from untreated primary hypertension, of which renal disease and some endocrine conditions are among the most salient. Even in its mild forms, hypertension is a progressive and lethal disease if left untreated.

In 2002, the USA National Center for Health Statistics revealed that the prevalence of hypertension was of 28.7% among Americans 20-74 years of age, and that 84.9% of women and 72.7% of men 75 years of age and older, have hypertension. The prevalence of hypertension among children reported by various studies, ranges from 5.4%-19.4%, and a recent study on 1066 elementary school children 8-13 years of age reveals that 21% of them suffer from high blood pressure.

Control of blood pressure is mainly achieved through regulation of the cardiovascular system by a variety of organs and systems external to the heart and blood vessels, such as the brain stem, medulla, spinal cord, autonomic nervous system and endocrine system. Some homeostatic control mechanisms of blood pressure begin to correct blood pressure deviations in just a fraction of a second, while others do so in up to a few minutes. Long term regulation of blood pressure implies controlling a set-point value along periods lasting several days to several months. Moreover, long term regulation of blood pressure focuses on controlling both fluid volume in the body, (especially blood volume) and blood sodium concentration. Long term regulation relies on renal function. However, there exists a transitional time zone between these two temporal domains of about 1 to 3 days, in which short term homeostatic regulation mechanisms gradually cease to have a significant effect, and the long term regulation mechanisms gradually become significant.

Despite the homeostatic control centers in the brain controlling cardiovascular and cardiorespiratory parameters, heart rate remains a remarkably quasi-independent biological factor. A number of findings strongly suggest that the body is unable to significantly effect long-term control of blood pressure:

Francis Bainbridge showed in 1915 that increasing venous return to the heart by fluid injection increases heart rate in dogs. This was attributed to a neural response now known as the Bainbridge Reflex. Subsequent studies revealed that a mechanical stretch of the sino atrial node pacemaker tissue gives rise to a positive chronotropic response even in denervated preparations, suggesting that the observed Bainbridge Reflex is not only neurally mediated, but is also partially encoded at the level of the sino atrial node pacemaker cells. A growing body of work has indicated that mechanical stretching of the right atrium by increased venous return may lead to an increase in heart rate that is independent of neural pathways, and which is attributed to local mechano-electrical feedback. Mechanical stretch of cardiac tissue almost instantly affects electro-physiology. This mechanical stretch effect produced by venous return to the right atrium is relatively small in normal subjects at rest, but is much more prominent during exercise, where it affects. Denervated heart transplant recipients show this mechanical stretch effect during exercise, which is consistent with the fact that respiratory sinus arrhythmia is not exclusively neurally mediated. Therefore, changes in heart rate associated with this local and quasi-instantaneous mechano-electrical phenomenon cannot be entirely regulated by feedback homeostatic control originating from neural and related hormonal cardiovascular centers.

At rest, the cardiac period is generally considered not to be defined by genetic factors but proportional to the ratio of the volume of the sino atrial node pacemaker cells to their surface area. Thus, an increase in heart rate occurs when the sino atrial node cells are deformed in a manner that increases their surface area while maintaining their volume constant, since this decreases the volume to surface area ratio. At rest, the diastolic pressure P_{d} is also considered to be highly dependent on dimensional factors, and therefore correlated to the resting heart rate.

The NHANES I Epidemiological Study on resting heart rate, was carried out to measure the resting heart rate, and to assess the association of resting heart rate with 25 variables, such as age, family income, education, poverty index, recreational exercise, body mass index, height, and hour of day, body temperature, systolic blood pressure (Ps), diastolic blood pressure_(Pd) and hemoglobin carried out with more than 11,000 persons aged 25-74 years old. Results of multiple linear regression analysis showed that the diastolic blood pressure has a consistent and highly significant independent association with heart rate, and is, by far the highest independent association among the variables studied, followed by a much lower association with hour of the day, body temperature and winter season. The association of heart rate and diastolic blood pressure remain significant even after controlling for smoking and multiple confounder variables.

A recent study with 269 healthy normotensive male medical students revealed that, when going from a low to a high dietary salt intake, there is an empirical need to consider 3 types of subjects in accordance with their physiological responses. There are salt-sensitive, salt-resistant and salt-inverse responder subjects. Salt-sensitive subjects (n:67) were characterized by a significant increase in systolic (5.3%) and diastolic (8.6%) blood pressure, while the heart rate increased slightly (1%). Salt-resistant subjects (n:136) were those for which almost no increase in systolic blood pressure was observed (0.4%), but whose diastolic blood pressure decreased (1.3%) and whose heart rate also decreased (2.5%). The inverse responders (n:66) were characterized by showing a decrease in both systolic (4.9%) and diastolic (8.9%) blood pressure, together with a notable decrease in heart rate (7.9%).

Thus, high dietary salt intake, in a first stage at least, increases blood volume due to water retention, resulting in an increase in venous return that should also be accompanied by an increase in heart rate and blood pressure. However, this is the direction of change taken by the cardiovascular system only for the salt-sensitive minority group (about 25% of the sample). In these experiments, the two factors involved in the long term regulation of blood pressure (blood volume and sodium concentration) have been affected. The results showed that most individuals compensate for the increase in blood volume following an elevation in sodium concentration by what seems to be a long term regulation mechanism of blood pressure leading to a reduction in blood pressure (the diastolic more than the systolic). This reduction in blood pressure is in direct proportion to the magnitude of the observed bradicardic effect. If the heart period increases (the heart rate decreases), the diastolic time period becomes longer and coronary perfusion is favored.

If the heart rate decreases, the systolic time period and the stroke volume increase. This increase in the heart period of a subject (while at rest and without changing his posture) represents an atypical bradicardic physiological phenomenon which can be explained in terms of responses of cardiopulmonary receptors to increased atrial stretching observed in salt resistant and inverse responders. For these particular populations, the work load of the heart is reduced in correlation with the extent of the bradicardic effect, reaching an average decrease of 12.2% in the inverse responders. The decrease in workload that took place in this population showed a direct correlation with the reduction in heart rate and also with blood pressure. In the long term, chronic increased salt intake translates into a resetting of pressure receptors that suppresses the short-lived atypical chronotropic physiological response to increased salt intake in most normal individuals.

Baroreceptor activity is the main factor involved in the short term regulation of blood pressure. In basal resting conditions, with cellular metabolism remaining constant, changes in total peripheral resistance are also governed by short term regulation of blood pressure, and mainly by sympathetic activity.

The arterial blood reaches the capillary network by means of the mechanical energy produced only by the contraction of the left ventricle, whereas, under normal conditions, the venous blood returning to the right atrium is influenced by three additional sources of mechanical energy, which are extrinsic to the cardiovascular system. The first source of mechanical energy is provided by the respiratory apparatus, by means of an alternating positive and negative pressure on the walls of the great veins in the abdominal and thoracic regions, as a result of the cyclical expansion and contraction of the thoracic cage during respiration. The second source of mechanical energy comes from the pumping effect produced by the leg muscles on the veins of the legs during locomotion, or the flexion extension movements of the legs. This phenomenon is made possible due to the upstream direction effect on venous blood produced by valves inside the veins of the legs. The third source of mechanical energy comes from the chaotic influence of the environment at large in the shape and form of gravitational and inertial forces acting upon the blood returning to the heart. These forces can cause significant changes in the distribution of the blood in the body as the body is subjected to changes in orientation relative to the direction of the gravitational vector. Thus, for example, when moving the body from a supine to a standing position, most of the venous blood falls into the veins of the legs and abdominal regions. Since these external forces are irregular, the dynamics of the circulatory system on its venous side is vulnerable to changes in the body's orientation. This can cause perturbations on the venous blood flow returning to the right atrium.

Venous return and atrial filling pressure are directly affected by these external factors. The atrial filling pressure is directly involved in building up the volume of blood inside the atria in a blood filling period culminating at the end of the ventricular systole, when the atrioventricular valves open and ventricular diastole starts. At this point in the heart cycle, the peak of the V wave of the venous pulse occurs, and atrial mechanoreceptors reach maximal stretch. Therefore, at this time, the atrial receptors are most sensitive to the external factors.

The external factors affecting blood circulation are substantially neutralized by compensatory short term homeostatic control mechanisms, mostly governed by negative feedback mechanisms. For example, when moving the body from a supine to a standing position, if a subject stands still for more than 10-15 minutes while standing, only about 15% of the blood will fall (mainly in the venous side) due to short term homeostatic control mechanisms. Most of these short term homeostatic control mechanisms involve receptors inside the blood vessels and the atria and ventricles of the heart, which can be involved in local, as well as in systemic regulations. If a malfunction were to occur in these short term homeostatic control mechanisms, the subject would experience an unpleasant health condition known as orthostatic stress in which he would be unable to remain standing still and may faint.

Regulatory factors influencing venous return are:
i. The Resting Metabolic Rate is expressed in Kcal produced per square meter of its body surface area per each hour. The diameter of arterioles changes in accordance with changes in the resting metabolic rate, leading to a corresponding change in total peripheral resistance.
ii. The degree of sympathetic outflow to the blood vessels in the cardiovascular system and particularly the sympathetic venous outflow. Increased sympathetic activity upon the smooth muscles of the veins decreases their compliance, producing a reduction of the volume of blood contained by the veins. In reducing this blood volume, some venous blood is expelled towards the right atrium, producing an increase of venous return and vice versa, when sympathetic venous outflow is reduced, the venous return also decreases.
iii. The blood volume contained inside the cardiovascular system. The blood volume decreases by fluid loss out of the blood vessels to the interstitial fluid and excretion by the kidneys, and increases through fluid intake. Assuming that venous compliance remains constant, when blood volume increases, venous return increases and vice versa.

U.S. Pat. No. 4,791,931 to Slate, discloses using blood pressure to regulate heart rate by implanting a transducer which, together with a pacemaker, operates as if it were an artificial baroreceptor reflex, in order to cause controlled changes in heart rate. In this way, short term control of blood pressure while maintaining resting heart rate may be obtained.

U.S. Pat. No. 6,141,590 to Renieri et. al, discloses a respiration modulated cardiac pacing system, by which variation in ventricular power output is minimized heart rate is made to increase during inspiration and decrease during expiration in order to maintain relatively constant left systolic stroke volume and also restoring respiratory sinus arrythmia in patient that may need it.

U.S. Pat. No. 4,686,987 to Salo et. al, discloses sensing systolic stroke volume by implantable means and by which a demand-type cardiac pacer can be controlled to change heart rate as a function of the sensed stroke volume. The stroke volume is maintained constant, which implies changing the heart rate in the same direction of the changes in venous return.

U.S. Pat. No. 5,913,879 to Ferek-Petril et. al, discloses an implantable vasovagal syncope detection device, where blood flow velocity and blood pressure data are obtained close to the tricuspid valve or in the superior vena cava, to identify a sudden reduction in peak values of either one of those parameters, which has been preceded by a sudden increase in sinus HR. This is an indicator of an upcoming vasovagal syncope.

U.S. Pat. Nos. 6,522,926 and 6,985,774 to Kieval et al., discloses implantable devices and methods for cardiovascular reflex control to reduce or increase blood pressure by activation or inhibition/dampening of baroreflex arterial signals, which are preferably those from the carotid sinus and/or the aortic arch.

U.S. Pat. No. 6,602,032 to Gavish et. al, discloses a non-invasive device for modifying, by voluntary activity, monitored physiological parameters obtained with transducers, such as the breathing rate.

U.S. Pat. No. 3,765,406 to Toole et. al, discloses a tiltable bed with an automatic control system that is used to obtain short term regulation of a physiological characteristic such as blood pressure, within pre-defined limits. The magnitude of the measured blood pressure selectively activates the motor of a tiltable platform or bed, to appropriately reposition the platform or bed to a new angle and affects blood pressure accordingly.

### SUMMARY OF THE INVENTION

The present invention provides a system and method for cardiovascular treatment and training. The system of the invention comprises a venous return device that induces a change in the venous return of an individual. The system of the invention also comprises a heart rate device that induces a change in the heart rate of the individual. The venous return device and the heart rate device are preferably under the control of a common processor configured to activate the venous return and heart rate device as required in any treatment.

The system of the invention may be implemented using any venous return device known in the art or to be developed in the future. Table 1 lists several known venous return devices and the effect (increasing or decreasing) they have on the venous return. The system of the invention may be implemented using any heart rate device known in the art or to be developed in the future. Table 2 lists several known heart rte devices and the effect (increasing or decreasing) they have on the heart rate. The venous return device and the heart rate device may be non-invasive or invasive devices.

The method of the invention comprises altering an individual's venous return and imposing a change in the individual's heart rate beyond any change in heart rate that occurred as a result of altering the venous return. When a subject changes postural position from standing to supine, an increase in venous return is produced, and the body will normally respond by increasing the stroke volume and cardiac output while decreasing the total peripheral resistance leading to a reduction in blood pressure and heart rate.

Without wishing to be bound by a particular theory, it is believed that the invention causes a change (increase or decrease) in the maximal diastolic volume attained by the atria in response to which cardiopulmonary receptors in the atria cause a change in blood pressure by signaling changes in renal function and in total peripheral resistance to effect a change in blood pressure and other cardiovascular system variables associated with blood pressure such as total peripheral resistance. It is also believed that changes in blood pressure are obtained via cardiopulmonary receptor responses of the atria to the resulting change in the atrial blood volume sensed by the atrial receptors, driving changes in renal function and in total peripheral resistance to effect the desired change in blood pressure and other cardiovascular system variables associated with the blood pressure and total peripheral resistance changes. It is further believed that use of the invention reinforces short term and long term regulation of blood pressure.

The system and method of the invention may be used, for example, in a program directed to achieving sustained, long term, regulation of blood pressure. Use of the invention may, in some cases, promote the neutralizing of environmental effects on the cardiovascular system. Accordingly, the present invention may be used in a program directed to the treatment of essential or secondary hypertension. In one embodiment of the present invention, the venous return is increased the heart rate is decreased in order to achieve a decrease in blood pressure. In another embodiment of the invention, the venous return is decreased and the heart rate is increased in order to achieve an increase in blood pressure.

The invention may also be used in a program aimed to affect total peripheral resistance, directed to slowing down the reduction in resting metabolic rate observed with age. Moreover, increases in resting metabolic rate could be obtained without the need of physical exertion, learning, voluntary cooperation or consumption of dietary supplements, so that the invention may be used as part of a program of fitness and health management by means of cardiovascular fitness equipment characterized by a ultra-low impact performance.

The invention may also be used in a program directed to increasing blood circulation in skin tissues. The increase in blood flow created by the invention can be directed, by techniques known in the art, to desired skin tissues by expanding the blood vessels in these skin tissues, to obtain an increase in the number of active arterioles and venules in the targeted skin. Such a program may be directed to produce a cosmetic effect on the selected skin surface.

When an increase in venous return is generated by the venous return device which leads to a bradicardic effect on the heart, the heart rate device may be used to further decrease the heart rate leading to a further increase in the systolic ejection time. In this case, an increased stroke volume may compensate, at least in part, for the increased volume of blood entering the right atrium. At the same time, the diastolic period may be prolonged, so that more coronary irrigation will be available to the heart ventricles. The tricuspid valve would remain closed for an extended time span due to the additional decrease in heart rate. Hence, the right atrium will be stretched further, even for the same or a decreased venous return. The increase in the volume of blood obtained by the invention inside the atrium is thus the result of the tricuspid valve remaining closed for a longer time due to the further decrease in the heart rate, which will be sensed by the right atrium receptors as if an increase in total blood volume has occurred. The left atrium will also be further stretched. The atrial receptors will increase release of atrial natiuretic factor and will send neural signals to control centers in the medulla and brain stem that all together will result in a reduction of total peripheral resistance, total blood volume and sodium content. The resulting decrease in blood pressure (diastolic and systolic), accompanied by a reduction in heart rate will be similar to the cardiovascular phenomenon observed in inverse responders following an increase of dietary salt intake. It follows from this that a hypotensive condition could be improved or corrected by reducing venous return, together with a tachycardiac effect on chronotropic cardiac activity.

The system and method of this invention can be implemented by embodiments referred to herein as “the basal regulatory mode” (BRM), and “the long-term regulatory mode” (LTRM). Use of these embodiments may produce long term changes in cardiovascular variables, including diastolic and systolic blood pressure similar to those produced by “inverse responders”, but without the need of increasing dietary sodium ingestion.

In one embodiment of the method of the invention, referred to herein as the “the long-term regulatory mode”, the method is delivered to an individual for a duration not exceeding about 30 minutes which is the time required by the short term homeostatic control of blood pressure to become fully active. Repeated applications of the method, each application being up to 30 minutes, and preferably between 5 and 10 minutes, in duration, and are preferably separated from each other by time intervals long enough to minimize the influence of the short term regulatory responses arising from the previous intervention. Preferably, a separation of at least six hours between consecutive applications of the method is used. The induced cardiovascular changes by the system and method of the invention cannot normally be fully compensated by short term homeostatic controls of blood pressure when the method is applied for a duration of up 30 minutes.

The long-term regulatory mode embodiment tends to minimize or avoid any correlation between the heart rate and venous return changes induced by the method of the invention, on the one hand, with other changes in these variables produced by short term homeostatic regulation of blood pressure, on the other hand. In this embodiment of the method, voluntary involvement, such as the “intent to move”, or an emotional arousal by the subject should preferably be avoided or minimized during the intervention duration. Therefore, in this embodiment, while forces and stimuli are directed to produce particular changes in the heart rate and venous return of a subject, the subject should remain at rest, and preferably in a passive condition, in order to minimize voluntary and emotional involvement.

Table 5 shows changes in heart rate and Table 6 show data from three published clinical studies in which the effect of various procedures on mean systemic arterial pressure was studied.

In Study A (Cardiovascular effects of static carotid baroreceptor stimulation during water immersion in humans; Bettina Pump et al; Am J Physiol Heart Circ Physiol 280:2607-2615, 2001), mean changes in heart rate and mean arterial pressure were measured after the following procedure:
Change in postural position, from seated to supine
Water Immersion while seated
Undergoing a neck suction procedure while seated (−22 mmHg)
Undergoing the combined effects of water immersion and neck suction while seated;

In study B: (Comparison of acute cardiovascular responses to water immersion and head-down tilt in humans; Makoto Shiraishi et al; J Appl Physiol 92: 264-268, 2002), mean changes in heart rate and in mean arterial pressure were measured after the following procedure:
Water Immersion while seated
Head Down Tilt of −6°,

In study C (Salt intake and left ventricular work load, Ulrike Schorr and Arya M. Sharma, Journal of Hypertension 18: 1721-1724, 2000), mean changes in heart rate and in mean arterial pressure were measured after the following procedure:

Heart rate and arterial pressure were measured in subjects following a low salt intake diet. The subjects were transferred to a high salt intake diet for seven days at which time heart rate and arterial blood pressure were measured again.

In studies A and B the postural seated position was used as the baseline condition for all the measurements and in study C, the postural supine position was used as the baseline condition for all measurements.

Based on the observed changes in heart rate and mean arterial pressure in the three studies, it can be estimated that the heart rate should be further reduced in the method and system of the invention by at least 5 to 10 beats per minute in order to obtain a significant decrease in blood pressure. A similar increase in heart rate would be necessary in order to obtain a significant increase in blood pressure. Nevertheless, to compensate for the very slow drift in the set-point values of blood pressure with age, as occurs in essential hypertension, only a minor further decrease in heart rate may be required, possibly a further decrease of only 2 to 3 beats per minute.

TABLE 5
Decreases in Heart Rate by different procedures on human subjects
Data from 3 clinical research studies

| | Heart Rate Decrease (in beats/minute) | | |
|---|---|---|---|
| Subject | Study A | Study B | Study C |
| Condition | (Condition: | (Condition: | (Condition: |
| During the | lasting 15 min) | lasting 30 min) | 7 days apart) |
| Measurements | n = 10 | n = 10 | n = 66 |
| Neck Section | 3 | | |
| (Seated) | | | |
| Water | 8 ± 2 | 6 ± 2 | |
| Immersion (Seated) | | | |
| Water | 10 ± 2 | | |
| Immersion and Neck | | | |
| Suction (Seated) | | | |
| Supine | 13 ± 1 | | |
| (−6⁰) Head down | | 11 ± 2 | |
| tilt | | | |
| Low salt intake | | | 4.7 ± 4.7 |
| followed by High salt | | | |
| intake | | | |
| (on Inverse | | | |
| Responders) | | | |

TABLE 6
Decreases in Mean Arterial Pressure by different procedures on
human subjects
Data from 3 clinical research studies

| | Mean Arterial Pressure Decrease (in mm Hg) | | |
|---|---|---|---|
| | Study A | Study B | Study C |
| Subject Condition | (Condition: | (Condition: | (Condition: |
| During Measurements | lasting 15 min) | lasting 30 min) | 7 days apart) |
| Neck section (Seated) | 3 ± 1 | | |
| Water | — | — | |
| immersion (Seated) | | | |
| Water immersion and | 4 ± 1 | | |
| neck suction (Seated) | | | |
| Supine | 7 ± 1 | | |
| Head Down Tilt (−6⁰) | | 9 ± 2 | |
| Low salt intake followed | | | 5.5 ± 4.5 |
| by High salt intake | | | |
| (on Inverse Responders) | | | |

TABLE 1
Examples of venous return device for use in the system of the invention.

| | | | Force and/or | | Possible venous return | |
|---|---|---|---|---|---|---|
| Example | Invasive | Non-Invasive | Stimulus can be | | directions of change | |
| N° | Means | Means | produced by, for example | Example of Use | + | − |
| 1.1 | | • | Gravitational force | Tilting table or bed. The subject's body is moved | • | • |
| | | | | head up and/or down | | |
| 1.2 | | • | Periodic compression- | External venous pump | • | |
| | | | decompression force | | | |
| 1.3 | | • | Hydrostatic Pressure | Filling a container with water up until the subject | • | |
| | | | | neck. Subject is to remain at rest | | |
| 1.4 | | • | Warming of legs | Wrapping the subjects legs with an insulated | • | |
| | | | | electrical heater sheet | | |
| 1.5 | | • | Cooling of legs | Placing the subject legs inside a mild refrigerating | | • |
| | | | | chamber | | |
| 1.6 | | • | IFV | Subject body placed upon a rotating platform (tilt | • | • |
| | | | | optional) | | |
| 1.7 | • | | Endoscopic venous pump | Positioned in the vena cava of a subject | • | • |
| 1.8 | • | | Small inflatable balloon | Locating a balloon inside the right atrium of a | • | |
| | | | | subject heart | | |

TABLE 2
Examples of heart rate device for use in the system of the invention.

| | | | | | Possible heart rate | |
|---|---|---|---|---|---|---|
| Example | Invasive | Non-Invasive | Force and/or Stimuli can be | | directions of change | |
| N° | Means | Means | produced by, for example | Example of Use | + | − |
| 2.1 | | • | Light | Directed to the subject's eyes to influence his/her supra- | • | • |
| | | | | chiasmatic nucleus in the brain | | |
| 2.2 | | • | Sound | Sounding to the subject's ears a low or high frequency | • | • |
| | | | | sound | | |
| 2.3a | | • | Mechanical/Pneumatic means | To produce in the subject a carotid baroreceptor | • | • |
| | | | | reflex | | |
| | | | | To produce in the subject a trigeminal cardiac | | • |
| | | | | reflex | | |
| 2.3b | | | | | | • |
| 2.4 | | • | Mechanical means | To produce a skin-pressure vegetative reflex on the subject | | • |
| 2.5 | • | | Electrical means | Implanting a cardiac pacer in the subject. The pacer will | • | • |
| | | | | become active only during the intervention time epoch | | |

Thus, in one of its aspects, the invention provides a system for cardiovascular treatment or training comprising:
(a) One or more venous return devices adapted for altering venous return in an individual;
(b) One or more heart rate devices adapted for altering a heart rate of the individual;
(c) a processor configured to:
   activate the one or more venous return devices at one or more predetermined levels for one or more predetermined time durations; and
   activate the one or more heart rate devices at one or more predetermined levels for one or more predetermined time durations.

In another of its aspects, the invention provides a method for cardiovascular treatment or training comprising, for each of one or more time durations:
(a) altering venous return in an individual at one or more predetermined levels; and
(b) altering a heart rate of the individual at one or more predetermined levels.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows a schematic diagram of a system for cardiovascular treatment or training comprising a venous return device and a heart rate device, in accordance with an embodiment of the invention;

FIG. 2a shows a venous return device for use in the system of FIG. 1 having a tiltable surface;

FIG. 2b shows a venous return device for use in the system of FIG. 1;

FIG. 3 shows a venous return device for use in the system of FIG. 1 comprising a pneumatic chamber;

FIG. 4 shows a venous return device for use in the system of FIG. 1 comprising a water chamber;

FIG. 5 shows a venous return device for use in the system of FIG. 1 comprising a leg heater;

FIG. 6 shows a venous return device for use in the system of FIG. 1 comprising a leg cooling chamber;

FIG. 7a shows a view from above of a venous return device for use in the system of FIG. 1 comprising a rotating circular platform, and FIG. 7b shows the device in a side view;

FIG. 8 shows a heart rate device for use in the system of FIG. 1 comprising a display screen;

FIG. 9a shows a heart rate device for use in the system of FIG. 1 comprising a device for applying pressure to the neck;

FIG. 9b shows a heart rate device for use in the system of FIG. 1 comprising a neck chamber;

FIG. 10 shows a heart rate device for use in the system of FIG. 1 comprising a device for applying bilateral chest oppression;

FIG. 11a shows a side view of a system according to FIG. 1 comprising the venous return device of FIG. 2 and the heart rate devices of FIGS. 8 and 10, FIG. 11b shows a side view of the system and FIG. 11c shows a top view of the system; and

FIG. 12 shows a flowchart for carrying out the method of the invention, in accordance with an embodiment of the invention.

### DETAILED DESCRIPTION OF EMBODIMENTS

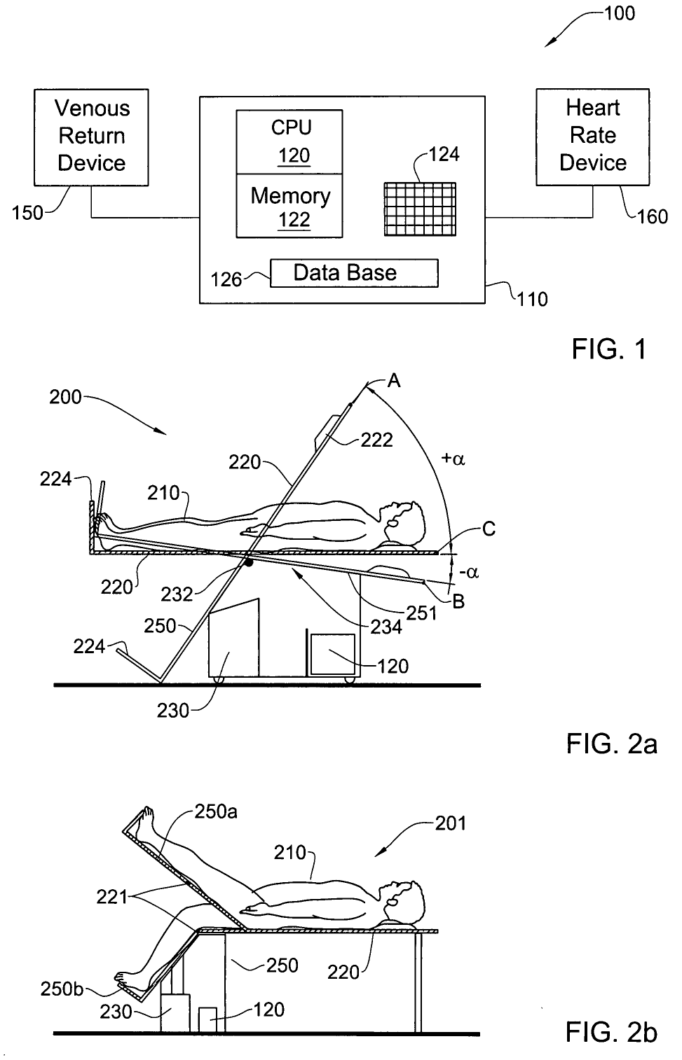

FIG. 1 shows schematically a system generally indicated by 100 for cardiovascular treatment or training in accordance with on embodiment of the invention. The system 100 comprises a venous return device 150 whose function is to alter the rate of venous return in a subject. The system 100 also includes a heart rate device 160 whose function is to alter the heart rate of the subject. The venous return device 150 and the heart rate device 160 are under the control of a control unit 110. The venous return device 150 and heart rate device 160 may be, for example, any one of the devices listed in Table 1 and Table 2, respectively. The control unit 110 includes a CPU 120, a memory 122, a database 126 and a user input device that may be, for example, a keypad 124. The input device can be used to input data to the memory 122 relating to the treatment to be carried out such as the direction and magnitude of the treatment (decreasing or increasing the venous return and heart rate), the duration of the intervention, information relating to the identity of the subject, and so on. The database 126 may be used to record responses of individuals to the treatment or training for later analysis. The CPU 120 is configured to operate the venous return device 150 and heart rate device 160 in accordance with the input instructions stored in the memory 122.

FIG. 2a shows a venous return device generally indicated by 200 for use as the venous return device 150 in one embodiment of the system 100. The venous return device 200 includes a tiltable surface 220, which may be a bed or table. An increase or decrease in venous return can be achieved by appropriate adjustment of the tilt angle of the surface 220. In FIG. 2a, a subject 210 is shown lying on the surface 220 which is in a horizontal position (tilt angle α=0° relative to the horizontal). A motor 230 is under the control of the CPU 120 that is used to adjust the tilt angle of the surface 220 according to data input by a user. The CPU 120 changes the subject's position from the initial supine position (α=0°) to a head up position (α>0°) or a head down position (α<0°), as specified for the treatment. For example in order to achieve an elevation in the venous return in relation to an head up position, the surface 220 may be brought to a tilt angle of about −8° so that the subject's trunk is lower than his legs. In order to achieve a decrease in the venous return, in relation to a supine position, the surface 220 may be brought to a tilt angle of about +58° so that the subject's trunk is above his legs.

A base 234 upon which the surface 220 pivots has a polygonal shape in order to provide additional support for the subject when the surface is maximally tilted. The head of the subject is supported by a pillow 222 and the legs of the subject are supported by a foot support 224. The table is connected to the base 234 at an axis 232, which allows tilting the bed and changing the tilt angle of the bed with respect to the ground. The base 234 provides physical support for the maximal head up (position A in FIG. 2a) and head down (position B in FIG. 2a). The system 200 also has two limit switches 250 and 251 which send signals to the CPU 120 when the surface has reached the position A or the position B respectively. The CPU 120 also controls the speed of the motor 230. Rotation of the surface 220 is preferably done at a constant angular velocity.

FIG. 2b shows a venous return device 201 in accordance with a second embodiment for use in the invention. The venous return device 201 includes a surface 220, which may be a bed or table, on which the subject 210 lies. The surface 221 has a first tiltable portion 250a and a second tiltable portion 250b, generally limited to the left leg region, and the right leg region, respectively of the subject. A motor 230 under the control of the CPU 120 is used to change the tilt of the tiltable portions 250 with respect to the horizontal. Each horizontal portion 250 may controlled individually, or both horizontal portions 250 may be activated in unison. Shifting the position of one or both the legs changes in venous return.

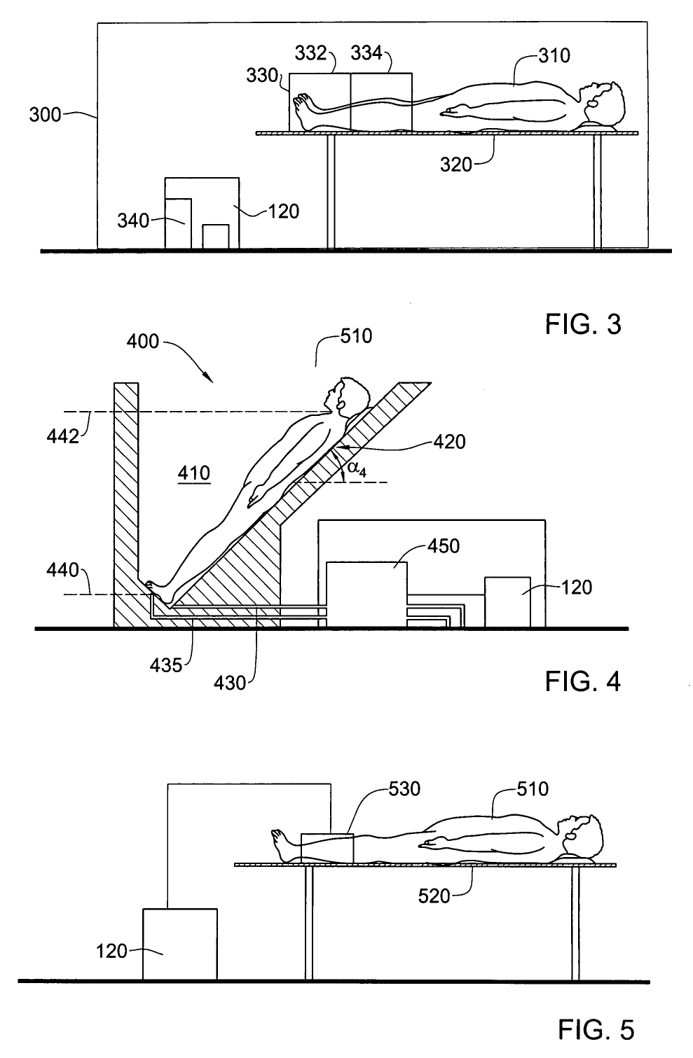

FIG. 3 shows a venous return device 300 for use in the system 100 in accordance with yet another embodiment of the invention. In the device 300, a pneumatic chamber or sleeve 330 serves as a venous return device. The pneumatic chamber 330 covers at least a portion of the subject's legs and applies cycles of alternating compression and decompression forces to the subject's legs, while the subject lies on a surface 320, which may be a bed or table. The pneumatic chamber 330 is associated and controlled by the CPU 120 which is configured to control the cyclically varying air pressure in the pneumatic chamber 330 by means of an air pump 340. In a preferred embodiment, the pneumatic chamber is divided into two or more cells 332 and 334 which are pressurized and depressurized in succession so as to apply a peristaltic pressure regime to the legs towards or away from the subject's trunk. Pneumatic chambers and sleeves are well known in the art.

FIG. 4 shows a venous return device 400 for use in the system 100 in accordance with another embodiment of the invention. In the device 400, a water chamber 410 serves as the venous return device by applying a hydrostatic pressure to a subject's body. A first pipe 430 is connected to the water chamber for conducting water into the chamber 410 during filling of the chamber, and a second pipe 435 for conducting water out of the chamber 410 during emptying. The flow of water into and out of the chamber 410 is driven by a motor-pump system 450, controlled by a the CPU 120. The subject lies on a tilted surface 420 in the water chamber 410 ata predefined angle α₄. The level of water into the chamber 410 is made to vary cyclically between a lowest water level 440 and a highest water lever 442. The highest water level 442 is preferably determined at the level of the neck of the subject, so as to completely submerge the subject's trunk. The filing and emptying cycle consists preferably of three time periods: a first time period during which the water level rises form the lowest level 440 to the highest level 442, a second time period during which the water remains at the highest level 442 and a third time period during which the water flows out of the chamber 410 to the lowest level 440. The duration of the first time period may be, for example, 6, 8, 10 or 12 minutes, the duration of the second time period may be 30 seconds and that of the third time period may be 1 minute. The increase in venous return as a result of water immersion is well known in the art.

The subject should preferably enter the water chamber 410 when the chamber is essentially empty of water in order to minimize voluntary movements of the subject that might occur when the subject leaps into a full container, which might have undesirable effects on the cardiovascular system.

FIG. 5 shows a venous return device 500 for use in the system 100, in accordance with another embodiment of the invention. The subject 510 lies on a surface 520, which may be a bed or table and a leg heater 530 is positioned to cover at least a portion of the subject's legs. According to certain embodiments, the leg heater 530 may be electric and under the control of the CPU 120 configured to control the temperature of the heating as well as the duration of the heating. A local and mild warming of the subject's legs, of about 4 to 8 degrees Celsius above body temperature will increase arterioles and small vessels lumen, ultimately inducing an elevation in venous return.

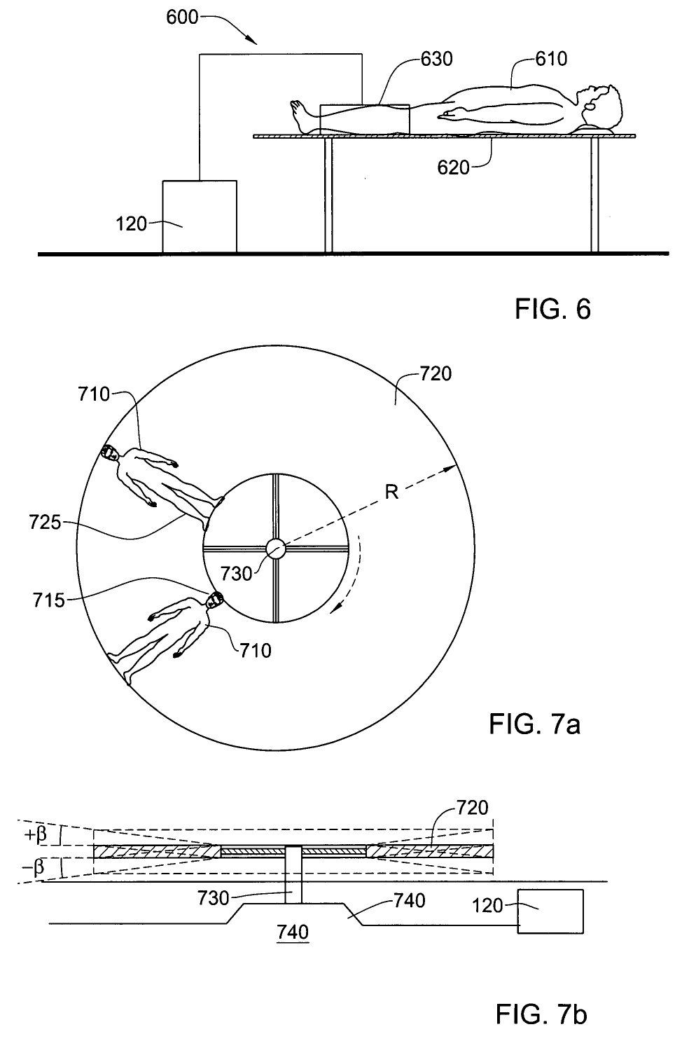

FIG. 6 shows a venous return device 600 for use in the system 100 in accordance with another embodiment of the invention. The subject 610 lies on a surface 620 that may be a bed or table. A leg cooling chamber 630 covers at least a portion of the subject's legs. The cooling chamber 630 is preferably electric and under the control of the CPU 120. The cooling in the chamber is preferably about 5 to 10 degrees Celsius below normal body temperature. Local cooling of the subject's legs induces a decrease in the venous return.

FIG. 7 shows a venous return device 700 for use in the system 100 in accordance with another embodiment of the invention. A rotating platform 720, shown from above in FIG. 7a and in a side view in FIG. 7b, generates centrifugal force on the body of a subject. The platform 720 has a radius R and is tilted by an angle β from the horizontal. The values of R and β may be determined as required in any application. The platform 720 rotates around an axis 730, for a predefined period of time. In order to induce a decrease in venous return, the subject 710 lies on the platform 720 in the position 715 in which the head is closest to the axis 730. The centrifugal acceleration acts upon the venous blood of a subject and propels the subject's venous blood flow towards the legs, resulting in a decrease in venous return. In order to induce an increase in venous return, the subject lies on the platform 720 in the position 725 with the feet closest to the axis 730. The platform 720 is rotated by means of a motor 740 which also serves to change the angle β of the platform 720. The motor is controlled by the CPU 120.

The venous return devices used in the embodiments of FIGS. 1 to 7 are non-invasive devices. The system of the invention may also be implemented using an invasive venous return device, such as an endoscopic venous pump. An endoscopic venous pump is positioned in the inferior vena cava and used to increase or decrease venous return. Another invasive device for influencing venous return consists of a small inflatable balloon that is placed inside the right atrium, some distance from the tricuspid valve. The balloon is inflated and deflated in coordination with the heart cycle, in a manner which is well known in the art. The CPU controls a time delay between, for example, pacemaker stimuli to the heart and inflation of the balloon, so that the balloon begins to inflate, for example, around the middle of the systolic ejection time. The balloon has an inflation-deflation cycle time that is preferably adapted to the particular requirements of the subject, but is typically about 150 msc.

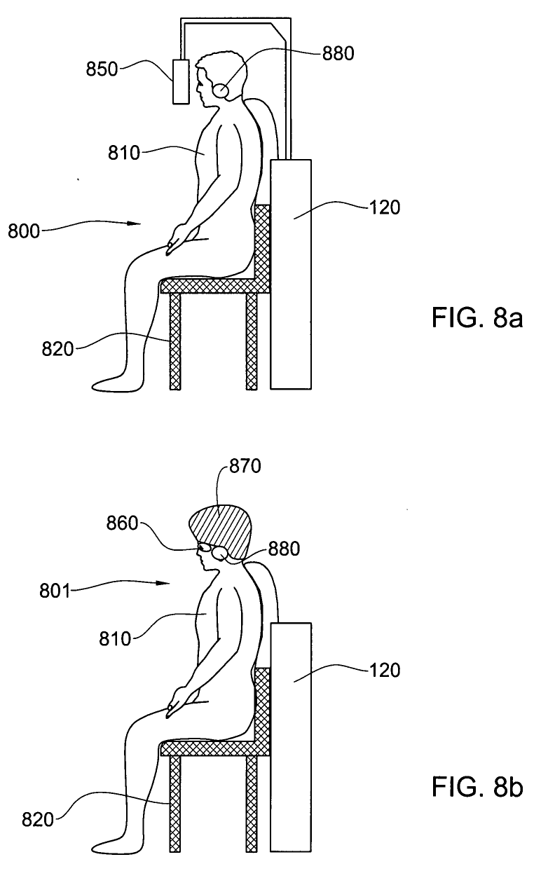

FIG. 8a shows a heart rate device 800 for use in the system 100 in accordance with one embodiment of the invention. A subject 810 sits on a seat 820 and observes a display screen 850. The screen is associated and controlled by the CPU 120 which is configured to display colors on the display screen 850 in a predefined sequence and timing to achieve the desired change in heart rate. In another embodiment 801 shown in FIG. 8b, a pair of goggles 860 that may be mounted on a helmet 870 displays colors to the subject. The goggles 860 are associated and controlled by the processor 120 as described for the embodiment of FIG. 8a. Headphones 880 which may be integral with the helmet 870 are associated and controlled by the CPU 120 which are configured to generate sounds in the headphones 880. The generated frequencies and melody may change during the time period of the treatment.

Table 3 presents a non-limiting example of a color sequence program for use in the devices 800 and 801. The sequence of colors in Table 3 is configured for inducing a bradicardic heart requested heart rate effect. For obtaining a bradicardic effect, the spectrum of light must be from green to violet with a higher proportion of blue and violet color. Each selected color is projected for a different duration of the treatment. Table 3 shows an example of a total exposure time of 6 minutes. One or more of the visual fields of the subject can be stimulated separately by the projected colors.

Light stimuli are capable of influencing the circadian rhythm of neuronal and hormonal activities, which in turn can regulate many different physiological processes, including those of the cardiovascular system, including heart rate. Visual exposure to blue/violet wavelengths slows down heart rate while exposure to red/yellow light increases heart rate. Sound below 400 Hz, as well as soft melodies, are known to induce a bradicardic effect, while sound frequencies above 800 Hz as well as fast rhythmic melodies may induce a tachycardic effect.

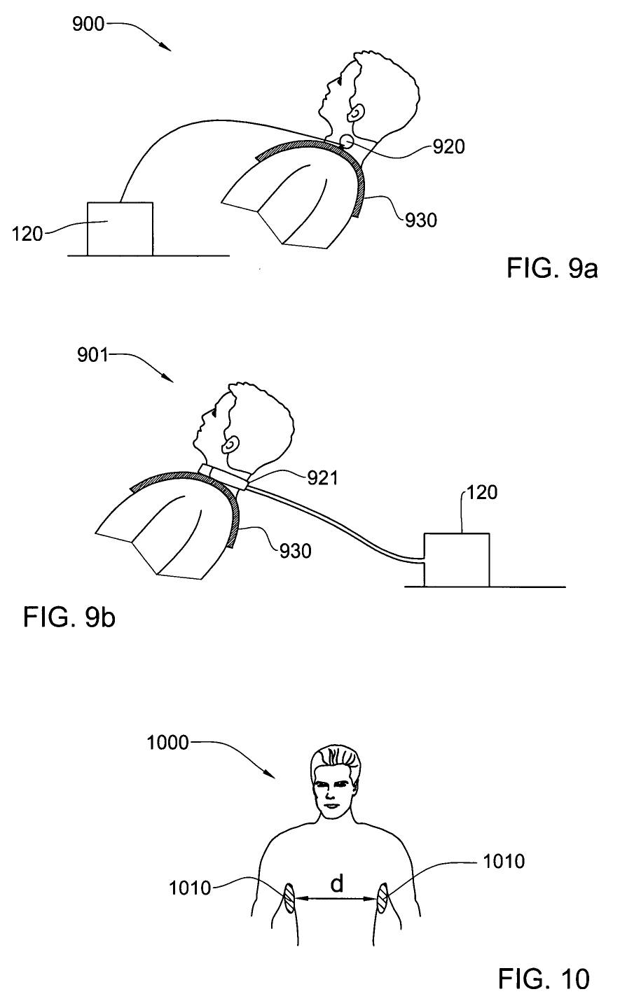

FIG. 9a shows a heart rate device 900 for use in the system 100 in accordance with another embodiment of the invention, in which mechanical pressure exerted on a spot of the carotid triangle on the neck, produces a lowering of the heart rate. When the required decrease in heart rate is around 5 to 10 beats per minute, only a light mechanical pressure is required. In the device 900 a presser 920 having a cylindrical or spherical shape is apposed to the carotic triangle and applies a pressure to the neck. The device 900 also has an ergonomic brace 930 adapted to be attached to the shoulders of the subject, to which the presser 920 is attached. The presser 920 is controlled by the CPU 120 which is configured to control the magnitude and duration of the applied pressure.

The presser 920 may comprise, for example, an electromagnetically activated piston that protrudes beyond a housing in order to apply a predetermined temporo-spatially array of pressure. In this way, desired mechanical pressure exerted on the neck of the subject can be regulated.

FIG. 9b shows another heart rate device 901 for use in the system 100 in accordance with another embodiment of the invention. A neck chamber 921 applies either positive or negative pressure to the neck surface by regulating the air pressure between the neck chamber 921 and the anterior surface of the neck. The pressure may vary, (e.g. sinusoidally) with time. A neck chamber, such as the chamber 921 is well known in the art for testing physiological characteristics of carotid baroreceptors in clinical research. Carotid baroreceptors can be artificially stimulated by neck suction and inhibited by neck compression. Neck suction reduces heart rate and is usually well tolerated by the subject. A reduction in heart rate by about 15 beats per minute can be obtained by a sinusoidally varying negative pressure having an amplitude of about −30 mm Hg and a frequency of 0.1 Hz, Furthermore, this possible form of heart rate control should be easily tolerable by the subject due its brevity (which is preferably less than 10 minutes).

The neck chamber 921 will typically embrace at least ⅔ of the anterior part of the neck. An ergonomic support 930 is adapted and attached to the shoulders of the subject, to minimize movements of the neck chamber 921 in relation to the anterior surface of the neck. Pressure changes in the neck chamber 921 are controlled by the CPU 120.

The invention may also be implemented by using the trigeminal cardiac reflex, originally reported in the literature as the oculocardiac reflex, and known as the Aschner Reflex. This reflex produces a decrease in heart rate by means of a slight compression of the eyeball. The Aschner Reflex is mediated by nerve connections between the trigeminal cranial nerve and the vagus nerve of the parasympathetic nervous system. Using the Aschner Reflex to obtain a 5 to 10 heart beats per minute reduction in the heart rate, should not be confused with the Aschner test, where a reduction by more than 10 heart beats per minute may be required. For this invention, only a light pressure exerted on the eyeballs (with eyes closed) is required, and for which a number of means can be devised.

FIG. 10 shows a heart rate device 1000 for use in the system 100 according to another embodiment of the invention. The device 1000 achieves a change in heart rate by applying bilateral chest compression. Bilateral chest compression at the axillary level of the chest brings about a decrease in heart rate. This bradicardic effect is known to be produced by the Skin Pressure-Vegetative Reflex. In the device 1000, a compression pad 1100 is placed bellow each of the axilla of a subject on both sides of the upper chest. The distance d between the two compression pads can be regulated using a variety of means known in the art. It should be noticed that when the chest is laterally compressed in its upper axillary region, abdominal breathing will be favored and an increase in the expiratory-inspiratory time ratio may also take place. This phenomenon will in turn also contribute to a decrease in heart rate by reducing the relative extent of vagal suppression during inspiration in addition to the bradicardic effect produced by the bilateral chest compression itself. When the device 1000 is used in the system 100 to reduce heart rate, together with the device 200 (FIG. 2a) for affecting venous return, the device 1000 will immobilize the subject's body during the up and down movements of the surface 220, greatly minimizing motion and emotional arousal of the subject. The subject is to remain at rest, preferably in a passive condition during the entire treatment.

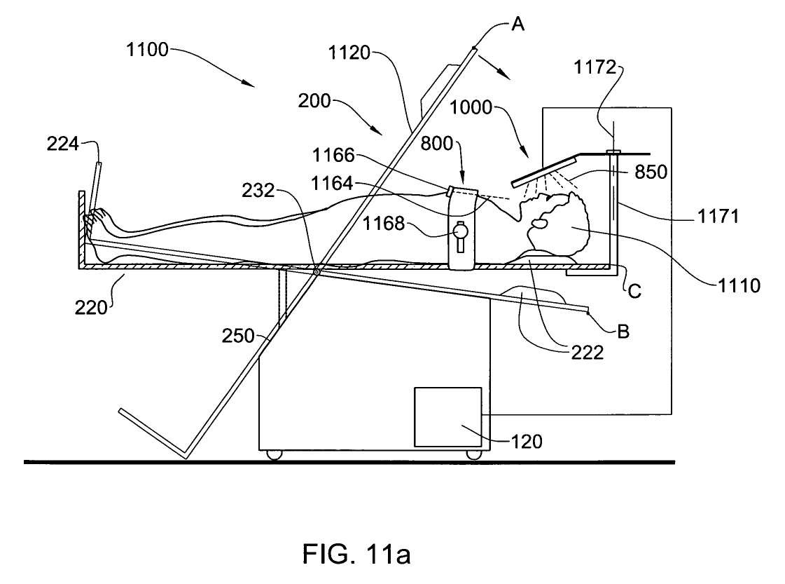

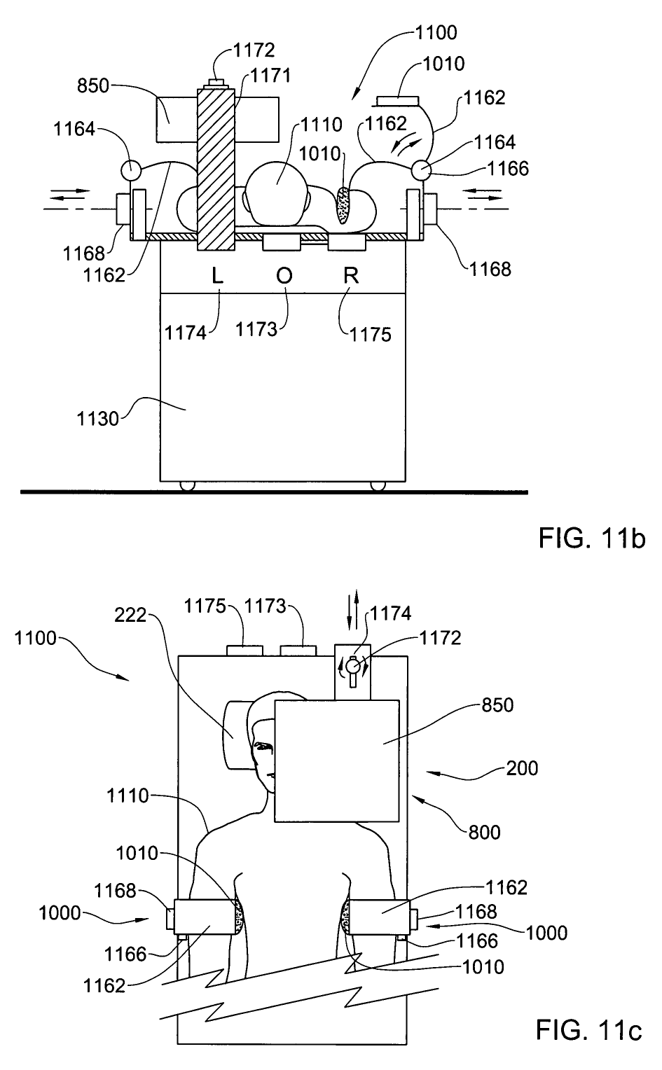

FIG. 11 shows an embodiment 1100 of the system 100 for cardiovascular treatment or training. The system 1100 is shown in a right side view FIG. 11a, in a top view in FIG. 11b, and in a view from above in FIG. 11c. The system 1100 includes as the venous return device 150 the venous return device 200 shown in FIG. 2a. The system 1100 also includes as the heart rate device 160 the device 800 shown in FIG. 8a as well as the heart rate device 1000 shown in FIG. 10.

FIG. 11c also shows the two compression pads 1010 of the heart rate device 1000

Each compression pad 1010 is fixed to a rigid support 1162 composed of two pieces. The support 1162 has an upper horizontal piece, capable of pivoting around an axis 1164 that can be fixed in the required position by a knob 1166, and a vertical displacement knob 1168 which can also be moved in a lateral direction for adjusting the pressure of the compression pads on the subject's chest.

The system 1100 also includes the screen 850 attached to a support column 1171. The screen 850 is used for displaying light stimulation to the subject 1110 in order to change the heart rate as previously explained. The support column 1171 is used for adjusting the position of the screen 850 in respect to the subject. The support column 1171 supports the screen 850 that can be adjusted in a center position O 1173, in a left position L 1174 or in a right position R 1175, relative to the subject. (The screen 850 is shown in the position L 1174 in FIG. 11) By selecting one of the two lateral positions (left or right) the light stimulation will reach the eyes of the subject primarily through either one of the left or right visual field. The screen 850 is attached to the support column 1171 by means of an axis 1172 in order to position the screen near the face of the subject for the treatment and to move the screen away from the subject after the treatment. The CPU 120 controls the duration and hue of the colors which are displayed on the screen. The sequence of colors displayed by the screen and the time interval for displaying each color are preprogrammed and stored in the memory 122.

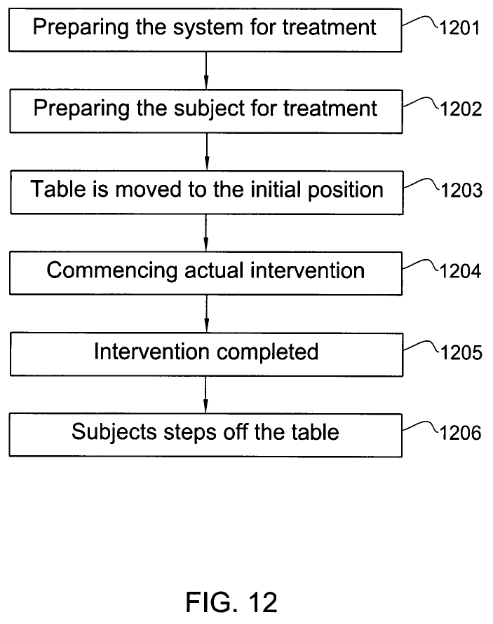

FIG. 12 shows a flowchart for carrying out the method of the invention using the system 1100. The first step 1201 is a preliminary stage for preparing the system before the treatment. This step includes preprogramming the CPU 120 with the data relating to the treatment. The information may include one or more of the following: the overall duration of the treatment, the duration of each part of the treatment. For example, the preprogramming information may include the time the subject is kept in each of the positions A and B, the angular velocity of the surface when switching from one position to another, the maximal angle for the head up position and the maximal angle for the head down position, and the color sequence program. In addition, during this step the position of the support column 1171 is adjusted to the appropriate position for the treatment. The next step 1202 includes preparing the subject for treatment. This stage can be performed together with step 1201. It is recommended for the subject to remain standing still and refrain, to the extent possible, from moving for about 5 minutes. This is done in order to allow some venous blood to flow towards the feet of the subject. This helps to attain better results in increasing venous return during treatment, as a sharper change in the direction of the blood is achieved. After standing still for 5 minutes the subject lays down on the surface 220. The compression pads 1010 are adjusted on both sides of the subject. The screen 850 is positioned in front of the face of the subject 1110. During the next step 1203, the surface 220 is moved to the initial position A by the operator. At this stage the system is ready to commence the actual intervention (step 1204). The device 200 is now activated and the surface 220 is moved from position A to position B at the designated angular velocity during which time the screen 850 displays the color sequence program. The screen 850 is turned off when the surface 220 moves from position B back to position A. When the treatment has been completed, the surface 220 is moved to the horizontal position (step 1205). The final step 1206 includes releasing the compressing pads 1010, moving the screen 850 away from the subject 1110 and allowing the subject 1110 to step down from the surface 220.

The system 1100 can also be used for preventing a long term decrease in blood pressure and/or promoting a long term increase in blood pressure. In these case, the method of the invention preferably comprises the following steps:
The subject is made to lie down for about 5 minutes in a supine position, preferably on the surface 220 at position C (where the tilt angle α=0)
The compression pads are not be utilized in these cases, since they tend to decrease heart rate.
An appropriate color sequence program is used for increasing heart rate. In order to achieve this tachycardic effect, light spectrum band to be used will be mostly from the red to the green colors, with a higher proportion of red color stimuli. A non-limiting example of a color sequence program to induce a tachycardic effect is shown in Table 4, for a treatment duration of 6 minutes:
Initial position is position B and not position A.
The treatment ends when the surface is at position A.

An invasive form of implementation can be devised by combining an endoscopic venus pump, with a cardiac pacer. For elevating venous return, an endoscopic venous pump for right-sided cardiac support can be utilized, such as those disclosed in U.S. Pat. Nos. 7,144,364 and 6,136,025 to Barbut et al. The pump is mounted in the interior of an expandable stent, which is releasably mounted on a distal end of a catheter. The pump can be inserted during surgery, for example using the femoral route in order to be positioned in the vena cava. The diameter of the pump should be smaller than the diameter of the femoral artery (0.5 to 1.0 cm) and with a length as short as 5 cm. The pump and the stent may be made of a biocompatible shape memory material, such as Nitinol. In this way it can be compressed before deployment and thereafter self-expanded. Alternatively, an angioplasty balloon in the catheter can be used to expand the stent intravascularly at the deployment site.

The pump may need to pump an amount of blood of up to about 0.5-1 liter/minute. This is about 10-20% of the amount of blood pumped by the heart for a venous return of 5 liter/minute. Assuming that the stroke volume remains constant, if for example, the heart rate is measured to about 70 beats/minute, the amount of blood being pumped by the pump will be equivalent to about 7-14 beats per minute.

TABLE 3
Example of a color sequence program for when a decrease in heart
rate is intended. Intervention time epoch: 6 minutes.

| | | Color time |
|---|---|---|
| | | interval |
| Condition | Color | (in minutes) |
| Starting together | Green | 1 |
| with intervention duration | | |
| | Blue | 1 |
| | Dark Blue | 2 |
| | Violet | 2 |

TABLE 4
Example of a color sequence program for when an increase in heart
rate is intended. Intervention time epoch: 6 minutes.

| | | Color time |
|---|---|---|
| | | interval |
| Condition | Color | (in minutes) |
| Starting together | Green | 1 |
| with intervention time | | |
| epoch | | |
| | Yellow | 1 |
| | Orange | 1 |
| | Red | 1 |
| | Strong Red | 2 |

1. A system for cardiovascular treatment or training comprising:
(a) One or more venous return devices adapted for altering venous return in an individual;
(b) One or more heart rate devices adapted for altering a heart rate of the individual;
(c) a processor configured to:
activate the one or more venous return devices at one or more predetermined levels for one or more predetermined time durations; and
activate the one or more heart rate devices at one or more predetermined levels for one or more predetermined time durations.

2. The system according to claim 1 wherein the processor comprises a memory storing data specifying values of parameters of a treatment.

3. The system according to claim 1 wherein the venous return device is adapted to increase the venous return.

4. The system according to claim 1 wherein the venous return device is adapted to decrease the venous return.

5. The system according to claim 1 wherein the heart rate device is adapted to increase the heart rate.

6. The system according to claim 1 wherein the heart rate device is adapted to decrease the heart rate.

7. The system according to claim 1 wherein the venous return device is non-invasive.

8. The system according to claim 1 wherein the venous return device is invasive.

9. The system according to claim 1 wherein the heart rate device is non-invasive.

10. The system according to claim 1 wherein the heart rate device is invasive.

11. The system according to claim 1 wherein the venous return device is selected from a tiltable surface, an external venous pump, a water tank, a leg heater, a leg cooler, a rotating platform, or a balloon adapted for insertion in an atrium.

12. The system according to claim 1 wherein the heart rate device is selected from a display screen displaying a predetermined pattern of light; a sound system producing a predetermined pattern of sound, a neck chamber, a pacemaker, a device for stimulating a carotid baroreflex, a device for stimulating a trigeminal cardiac reflex, and a device for producing a skin pressure vegetative reflex.

13. The system according to claim 1 wherein the venous return device is adapted to increase the venous return and the heart rate device is adapted to decrease the heart rate.

14. The system according to claim 1 wherein the venous return device is adapted to decrease the venous return and the heart rate device is adapted to increase the heart rate.

15. A method for cardiovascular treatment or training comprising, for each of one or more time durations:
(a) altering venous return in an individual at one or more predetermined levels; and
(b) altering a heart rate of the individual at one or more predetermined levels.

16. The method according to claim 15 wherein the venous return is increased.

17. The method according to claim 15 wherein the venous return is decreased.

18. The method according to claim 15 wherein the heart rate is increased.

19. The method according to claim 15 wherein the heart rate is decreased.

20. The method according to claim 15 wherein the venous return is altered non-invasively.

21. The method according to claim 15 wherein the venous return is altered invasively.

22. The method according to claim 15 wherein the heart rate is altered non-invasively.

23. The method according to claim 15 wherein the heart rate is altered invasively.

24. The method according to claim 15 wherein the venous return is altered by using one or more of the following devices: a tiltable surface, an external venous pump, a water tank, a leg heater, a leg cooler, a rotating platform, or a balloon adapted for insertion in an atrium.

25. The method according to claim 15 wherein the heart rate is altered by one or more of the following devices: a display screen displaying a predetermined pattern of light; a sound method producing a predetermined pattern of sound, a neck chamber, a pacemaker, a device for stimulating a carotid baroreflex, a device for stimulating a trigeminal cardiac reflex, and a device for producing a skin pressure vegetative reflex.

26. The method according to claim 1 wherein the venous return is increased and the heart rate is decreased.

27. The method according to claim 1 wherein the venous return is decreased and the heart rate is increased.

28. The method according to claim 15 wherein the time durations are up to 30 minutes.

29. The method according to claim 15 wherein consecutive time durations of the method are separated by a duration of non-treatment of at least 30 minutes.
